SEQUENCE LISTING (3 sequences)

SEQ ID NO: 1 - DNA, 48 bp, Artificial Sequence
  MAP - aptamer sequence
cctgccacgctccgctcactgacctgggggagtattgcggaggaaggt

SEQ ID NO: 2 - DNA, 12 bp, Artificial Sequence
  QDNA - Dabcyl labeled nucleotide
cccaggtcagtg

SEQ ID NO: 3 - DNA, 15 bp, Artificial Sequence
  FDNA - Fluorescein labeled nucleotide
gcggagcgtggcagg

(19) United States
(12) Patent Application Publication
(10) Pub. No.: US 2006/0275854 A1
(43) Pub. Date: Dec. 7, 2006

(54) Method for monitoring reactions in real time

(76) Inventors: Yingfu Li, Dundas (CA); Razvan Nutiu, Ontario (CA)

Correspondence Address: FISH & RICHARDSON PC, P.O. BOX 1022, MINNEAPOLIS, MN, 55440-1022, US

(21) Appl. No.: 10/562,677

(22) PCT Filed: Jun. 29, 2004
(86) PCT No.: PCT/CA04/00966

§ 371 Date: May 16, 2006

Related U.S. Application Data
(60) Provisional application No. 60/483,636, filed on Jul. 1, 2003.

(51) Int. Cl. C12Q 1/66; G06F 19/00

(52) U.S. Cl. 435/8; 702/19

(57) ABSTRACT

Methods for monitoring chemical reactions in real time are provided. The methods involve the use of a signaling aptamer that has a different affinity for the substrate and the product of the chemical reaction. The conversion from substrate to product is detected a change in signal, in particular a change in fluorescent signal. The methods of the present invention are also useful to measure enzyme activity and to screen for enzyme inhibitors.

### FIELD OF INVENTION

The present invention relates to signaling aptamers for enzyme activity monitoring and inhibitor screening.

### BACKGROUND OF THE INVENTION

Aptamers are nucleic acids with ligand-binding capabilities that are isolated from random-sequence nucleic acid pools. Aptamers may comprise RNA, DNA or nucleotide derivatives. Several reports have described fluorescence-based signaling aptamers and signaling ribozymes and deoxyribozymes for the detection of small molecules and proteins in solution. There remained however a need for dynamic reporters that can be used to monitor a chemical reaction in real time as well as reporters that can be used to screen for inhibitors of a chemical reaction.

Enzymes are abundant in nature. They catalyze a large array of chemical transformations and act on a broad scope of substrates. In addition, many artificial enzymes including catalytic antibodies, ribozymes and DNAzymes have been generated. This broadens the scope of the use of enzymatic reactions in assays and creates new opportunities for novel applications. The important biological roles of enzymes and their increasing utility in a variety of applications place a significant need for convenient analytical methods to both characterize specific enzyme properties and to detect enzymatic activities. Conventional methods for analyzing enzymatic reactions often involve the use of sophisticated instrumentation such as high performance liquid chromatography (HPLC). Although those methods are accurate, the analytical processes are often time-consuming and difficult to adapt to unconventional applications such as high throughput robotic screening. Therefore, methods that are capable of monitoring enzyme activities conveniently, particularly in real time, are highly desirable.

### SUMMARY OF THE INVENTION

The present invention exploits the use of signaling aptamers as real-time probes to report enzyme activities. Several reports have described the use of signaling aptamers for the detection of small molecules and proteins in solution. An aptamer binds to a target and may have different affinities for related targets. The present invention discloses, for the first time, nucleic acid aptamers that can be used to monitor enzymatic reactions in real time.

In one aspect, the present invention provides a method for monitoring a chemical reaction. The method comprises providing a signaling aptamer having differential affinities for a substrate or substrate target and a product target. Intermediate targets may also be detected. The term “substrate” is used herein to refer to any substance that is acted on in a chemical reaction. The term “product target” refers to a product of a chemical reaction. The signaling aptamer is modified with a reporter molecule that provides a signal when the aptamer binds to a target.

In one aspect, the method of monitoring a chemical reaction comprises interacting a substance with a signaling aptamer capable of binding to the substance and monitoring for a change in the amplitude of the signal, wherein a change in amplitude is indicative of transformation of the substance and disruption of aptamer binding to the substance.

In another aspect, the method comprises incubating substance A in the presence of a signaling aptamer that has a first affinity for substance A and a second, different affinity for product B, determining the amplitude of the signal based on the affinity of the aptamer for substance A, providing conditions favourable for the conversion of substance A to product B, and monitoring for a change in amplitude of the signal.

In a preferred embodiment, the signaling aptamer comprises a fluorophore and a quencher in close proximity. Upon binding to a target the quencher is separated from the fluorophore and a fluorescent signal is generated. The signaling aptamer generally expresses different affinities for different targets (i.e. substrate, intermediate, product). Thus, the signal generated will be either increased or decreased as the reaction proceeds.

In a particularly preferred embodiment, the signaling aptamer is a signaling aptamer complex (SAC). The SAC comprises a target binding oligonucleotide duplexed with a fluorophore modified oligonucleotide and a quencher modified oligonucleotide.

In another aspect, the method comprises incubating a signaling aptamer in the presence of a substrate, determining a baseline fluorescence, adding a test sample and monitoring for a change in fluorescence. A change in fluorescence is generated as the substrate is converted to product.

In one embodiment, the aptamer recognizes the product target with a greater affinity and thus an increase in fluorescence is seen as the reaction progresses.

In another embodiment, the aptamer has a greater affinity for the substrate and a decrease in fluorescence is seen.

In another preferred embodiment, the chemical reaction is an enzymatic reaction. In a further preferred embodiment, the method is useful to detect the activity of a phosphatase, deaminase, adenyl cyclase or phosphodiesterase.

In yet another preferred embodiment, the chemical reaction is a phosphorylation reaction.

In another aspect, a method for the detection of an enzyme is provided. The method comprises incubating a substrate with a signaling aptamer having an affinity for the substrate, measuring the baseline fluorescence signal, adding an enzyme test sample and monitoring for a change in fluorescent signal, wherein a change in fluorescent signal is indicative of enzyme activity.

In a preferred embodiment an assay for the detection of phosphatase is provided. A signaling ATP aptamer is incubated in the presence of AMP, the baseline fluorescence is recorded, a test sample is added and the change in fluorescence is measured. An increase in fluorescence is indicative of the presence of a phosphatase.

In a preferred embodiment, alkaline phosphatase activity is monitored using a signaling aptamer that displays a sequential, differential fluorescent signal amplitude as ATP is converted to ADP, ADP is converted to AMP and AMP is converted to adenosine by alkaline phosphates (ALP). A method of screening for ALP inhibitors comprises comparing the sequential change in fluorescent intensity in the presence of a potential inhibitor as compared to the control reaction.

In a further aspect of the invention, a method of screening for an enzyme inhibitor is provided. The method comprises obtaining a signaling aptamer that exhibits a differential affinity and therefore a different fluorescent signal in the presence of the enzyme substrate versus the product of the enzyme reaction and establishing a threshold change in the amplitude of the fluorescence signal that is indicative of a conversion from the substrate to the product. The method further comprises introducing a potential inhibitor into the reaction, determining a change in the amplitude of the fluorescent signal and comparing the amplitude change to the change in amplitude in the absence of the inhibitor. The efficacy or strength of the inhibition can be determined by comparing the fluorescent signal generated in the presence of the inhibitor to a standard curve.

It is clearly apparent that in all the methods of the present invention, the order in which the components are added can be varied.

In another aspect of the invention, enzyme inhibitors identified by the method are provided. The use of the identified inhibitors in diagnostics and therapeutics is also encompassed.

The present invention also provides for the identification and use of enzyme inhibitors.

New uses for known compounds, such as aptamers and small molecules, are provided using the methods of the present invention.

A method and assay system for the detection and quantitation of an enzyme are also provided. The method comprises establishing a standard curve of fluorescent values generated by a signaling aptamer in response to predetermined amounts of an enzyme, reacting a test sample with the signaling aptamer, determining the fluorescent signal generated and comparing that signal to the standard curve to determine the amount of enzyme activity in the test sample.

In another aspect of the invention, an assay system is provided. The assay system comprises a signaling aptamer which has a target binding domain flanked by a fluorophore and a quencher. The aptamer binding domain has differential affinities for a substrate and a product. Control reagents are also provided. The assay system may be provided in the form of a kit.

In a preferred embodiment, a reaction monitoring kit is provided. The kit comprises a substrate and a signaling aptamer capable of binding to the substrate.

In another embodiment, a kit for detecting enzyme activity is provided. This kit comprises a signaling aptamer, an aptamer target and a control enzyme. The kit can also be used to screen for enzyme inhibitors.

### BRIEF DESCRIPTION OF THE DRAWINGS

These and other features of the invention will become more apparent from the following description in which reference is made to the appended drawings wherein:

FIG. 1A illustrates the structure of an exemplary signaling aptamer complex;

FIG. 1B demonstrates the ability of the aptamer complex of FIG. 1A to distinguish between adenosine, AMP, ADP, and ATP;

FIG. 1C illustrates the fluorescent signaling profile of the signaling aptamer in the presence of various amounts of adenosine, AMP and AMP treated with calf ALP;

FIG. 2 demonstrates the fluorescence intensity differential of the aptamer, ΔF, vs target (AMP or adenosine) concentration.

FIG. 3 demonstrates the enzymatic conversion of AMP to adenosine.

FIG. 4A illustrates the signaling profile of the aptamer in the presence of variable amounts of calf intestine alkaline phosphatase;

FIG. 4B demonstrates the signaling rate constant vs. concentration of alkaline phosphatase;

FIG. 5A illustrates the effect of various small molecules on alkaline phosphatase activity;

FIG. 5B demonstrates the results of inhibition assays performed in a 96-well assay format;

FIG. 6 illustrates the effect of an adenosine deaminase inhibitor on enzyme activity as measured by aptamer binding affinity.

### DETAILED DESCRIPTION

The present invention relates to a method of monitoring chemical reactions using nucleic acid aptamers which have different affinities for various substrates and products. Aptamers are oligonucleotides that have been selected for specific binding to a variety of molecular targets including proteins and small molecules.

Aptamers that have differential affinities for substrates and products are used to monitor enzymatic reactions in real time. The aptamers can be selected using a variety of approaches. For example, an aptamer can be selected for specific binding to a particular target using known in vitro selection techniques such as those described in the following references: Tuerk, C. and Gold, L. (1990) Science 249, 505-510; Ellington, A. D. and Szostak, J. W. (1990) Nature 346, 818-822; Famulok, M. (1999) Curr. Opin. Struct. Biol. 9, 324-329. Each of these documents is hereby incorporated by reference.

An aptamer with specificity for a particular target can also be selected using the aptamer selection technique described in PCT/CA2004/000482 that is incorporated by reference herein.

The aptamers which are useful in the present invention are signaling aptamers that have been engineered to incorporate reporter molecules, such as chromophores, fluorophores, isotopes and metals. Standard techniques can be used to label the aptamers.

The signaling aptamer may be a molecular beacon as described in Tyagi and Kramer, 1996, incorporated herein by reference. A molecular beacon is an oligonucleotide modified with a fluorophore at one end and a quencher at the other end. In the absence of the target, the molecular beacon adopts a closed state stem-loop configuration in which the fluorophore and quencher are in close proximity and the fluorescence is quenched. In the presence of target, the beacon adopts an open state where the fluorophore and quencher are separated and a fluorescent signal is generated.

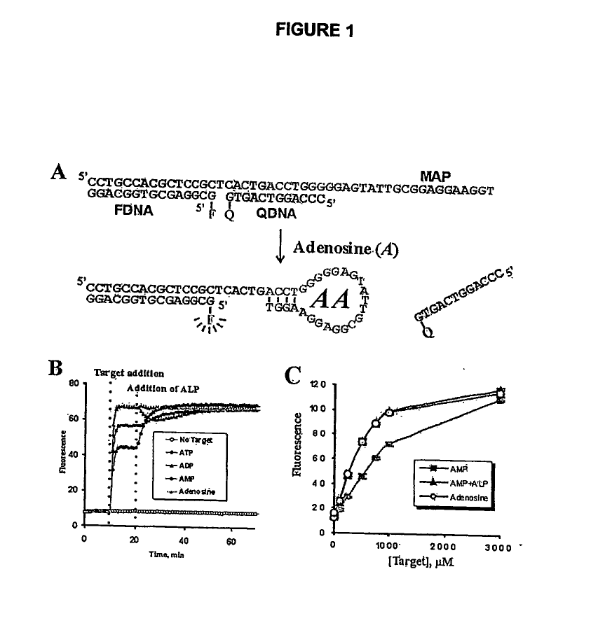

Alternatively, the signaling aptamer is preferably a signaling aptamer complex as described in PCT application CA03/00086, incorporated herein by reference. In one example, a quencher modified oligonucleotide which has a sequence complementary to part of the target-binding sequence hybridizes to the aptamer sequence and another oligonucleotide which is fluorophore-modified binds to a non-target-binding sequence of the aptamer. In the absence of target the quencher-modified oligonucleotide and the fluorophore-modified oligonucleotide are in close proximity and the fluorescent signal is quenched. In the presence of target, they become separated as the quencher modified oligonucleotide is displaced and a fluorescent signal is generated. It is clearly apparent that the signaling aptamer complex could have the fluorophore oligonucleotide hybridized to the target binding sequence and vice versa. An example of this type of signaling aptamer complex is illustrated in FIG. 1 and discussed further in Example 1.

The present invention is based on the discovery that some aptamers have differential affinities for specific substrate targets and product targets. During the course of a chemical reaction, a substrate target is converted to a product target. The term product is used broadly herein to refer to any transformed substrate resulting from a reaction. Since the aptamer has a different affinity for the substrate target than for the product target, the conversion from a substrate to a product by can be measured by a change in the intensity of the fluorescent signal as binding of the aptamer to the substrate is disrupted.

A signaling aptamer can be used to report a simple chemical reaction, A→B, in real time, if the signaling aptamer exhibits different levels of fluorescence upon binding to A and B. For example, if the signaling aptamer has a greater affinity for B, there will be a higher fluorescence readout for B than for A. The transformation of A to B can be conveniently monitored by following the fluorescence intensity increase upon binding of the signaling aptamer. On the other hand, if the aptamer has a greater affinity for A, there will be a decrease in the fluorescent signal as A is transformed to B. If an enzyme mediates the chemical reaction, a fluorescent aptamer reporter according to the present invention permits real-time monitoring of the enzyme's activity. The signaling aptamer complex can also be used in assays to screen for inhibitors of the enzyme activity.

According to the invention, a chemical reaction in which a substrate is converted to a product is monitored by first obtaining a signaling aptamer that has a different affinity for the substrate and the product. It should be understood that a product of one reaction can be a substrate for a subsequent reaction. For example, there can be a series of reactions in which substance A is converted to substance B, then substance B is converted to substance C, followed by conversion of substance C to substance D. The signaling aptamer distinguishes between the various substances based on differences in affinity and thus the transition from one substance to another is followed by a change in the amplitude of the signal. Typically, a baseline signal for the signaling aptamer is determined. When the substrate target is added, there is an increase in fluorescence as the signaling aptamer binds to the target and the quencher and fluorophore are separated. If a signaling aptamer complex is used, the quencher modified oligonucleotide is displaced from the duplex it had formed with the aptamer binding sequence. A fluorescent signal is generated and when an enzyme is introduced, the substrate is converted to a product. Since the aptamer has a different affinity for the product, there will be a change in fluorescence as the constant rate of aptamer complex bound will be either increased or decreased. In this manner, the conversion of substrate to product can be monitored in real time. Signaling aptamer complexes are particularly useful for monitoring chemical reactions due to their ability to induce several fold increase in fluorescence upon target binding. Thus, they can provide a sensitive readout. The method of the invention can be used to follow many different types of chemical reactions. For example, addition, transfer or removal of a functional group can be detected. Some examples of enzymatic reactions that can be monitored include those mediated by a phosphatase, a deaminase, an adenyl cyclase and a phosphodiesterase. It is clearly apparent that any chemical reaction where a signaling aptamer has a different affinity for the substrate and the product can be monitored using the methods of the present invention.

The invention also provides a method of detecting enzyme activity. The presence, in a test sample, of an enzyme capable of converting a substrate to a product can be determined by obtaining a baseline signal for the signaling aptamer or signaling aptamer complex, adding the substrate and determining the amplitude of the fluorescent signal. The test sample is then added and the fluorescent signal is monitored. A change in fluorescent signal amplitude is indicative of the presence of an enzyme converting the substrate to a product. The change may be an increase or a decrease in amplitude depending on the relative affinity for the substrate and the product.

Enzymatic activity can be quantified by comparing the amplitude of a change in fluorescence with a standard curve. The standard curve is obtained by incubation of a known amount of substrate with predetermined amounts of the specific enzyme to be detected. It is apparent that the signaling aptamer complexes can also be used as sensitive reporters to distinguish between closely related compounds.

The present invention also provides a method of screening for enzyme inhibitors. An enzyme assay can be set up as described above. The optimal substrate and enzyme concentrations to get a peak change in fluorescence amplitude are determined. A test compound is added and if the compound inhibits the enzyme, the characteristic change in fluorescence is not seen. The relative efficacy and required dose of the inhibitor can be determined by evaluating the degree of inhibition. The method can be used to screen for inhibitors of various enzymes.

The invention can be demonstrated using an ATP aptamer signaling complex. The structure-switching ATP reporter shown in FIG. 1A is able to generate different fluorescence signals when adenosine and its 5′-phosphorylated species are used as targets. For example, at 750 μM of adenosine, AMP, ADP (adenosine 5′-diphosphate), and ATP (adenosine 5′-triphosphate), the signaling aptamer produced a fluorescence intensity increase of 8.1, 5.3, 8.3, and 6.9-fold, respectively, over the background reading as shown in FIG. 1B. Each target was added after the signaling aptamer mixture was incubated for 10 min, indicated by the first dashed line. These observations demonstrate that this signaling aptamer is well suited as a reporter for nucleotide-dephosphorylating enzymes such as alkaline phosphatase (abbreviated as ALP), which is known to remove the 5′-phosphate groups from ATP, ADP, AMP and ultimately convert each of them into adenosine.

This efficiency of the reporter system was demonstrated by adding ALP to each target-aptamer mixture 10 minutes after the target addition as shown by the second dashed line in FIG. 1B. In the adenosine solution, the addition of ALP did not result in any intensity change as would be expected since no chemical reaction was expected to occur. The addition of ALP did generate a rapid fluorescence intensity increase in the AMP solution, as the AMP was converted to adenosine for which the aptamer had a higher affinity. ALP also promoted an intensity change in the ADP solution. The intensity initially decreased, then slowly recovered to the original level. Since ADP contains two phosphate groups (α and β phosphates), two dephosphorylation reactions should occur. The initial intensity drop was consistent with the removal of the β-phosphate (generation of AMP from ADP, coupled with fluorescence intensity decrease); the eventual intensity recovery is due to the accumulation of AMP and the subsequent removal of the α-phosphate (generation of adenosine from AMP, coupled with fluorescence enhancement). When added to the ATP solution, ALP induced a slow fluorescence increase. This is consistent with the signaling behaviours associated with three dephosphorylation reactions: the removal of the γ-phosphate from ATP (generation of ADP, accompanied by fluorescence increase), the removal of the β-phosphate (generation of AMP, accompanied by fluorescence decrease), and the removal of the α-phosphate (generation of adenosine, accompanied by fluorescence increase). It appeared that the fluorescence gain (resulting from the first and the third reactions) always surpassed the fluorescence loss (associated with the second reaction) during the entire course of incubation. These observations indicate that the ATP reporter can be utilized as a unique real-time probe to report the reaction catalyzed by ALP.

The AMP-aptamer system was selected to demonstrate that a signaling aptamer can be used to quantify enzyme activities according to the method of the present invention. Initially, titration curves for adenosine (circles), AMP (squares) and AMP treated with ALP (triangles) (FIG. 1C) were established. As expected, AMP treated with ALP gave the same fluorescence vs. concentration curve as adenosine.

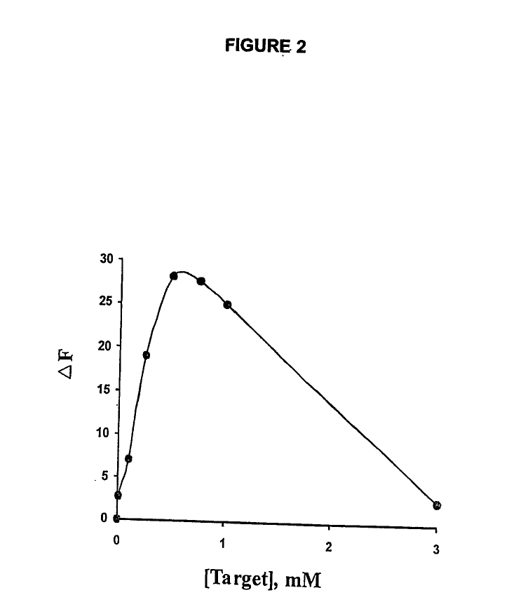

To establish the AMP concentration that generates the largest signal for AMP-to-adenosine transition, the fluorescence intensity differential (ΔF, defined as F _{adenosine}-F_{AMP}) was determined as a function of the target concentration. A bell-shaped curve was observed with ΔF peaking at a target concentrations around 500-1000 μM as shown in FIG. 2.

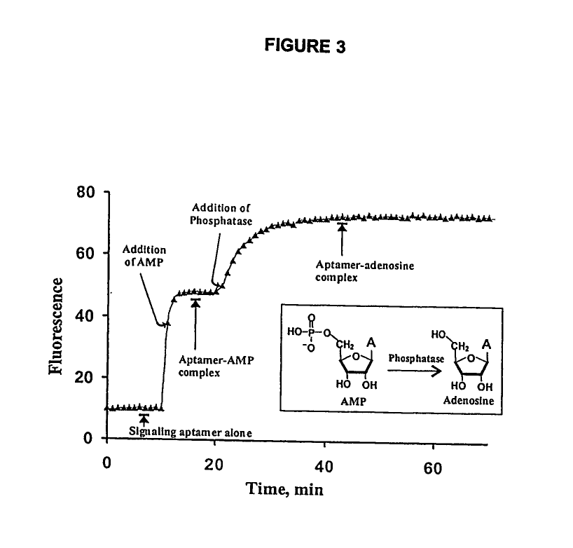

The conversion of adenosine 5′-monophosphate (AMP) into adenosine by alkaline phosphatase (ALP) was used as a model reaction to demonstrate enzyme activity. This specific reaction is shown in greater detail in FIG. 3. A structure-switching signaling DNA aptamer was used as the fluorescent reporter. The signaling aptamer, exhibiting a higher affinity for adenosine than for AMP, generates a unique two-leg signaling profile. The first leg of the signal profile is generated upon addition of AMP (indicative of the formation of the substrate-aptamer complex) and the second leg is generated upon addition of ALP (reporting the enzymatic conversion of the substrate to the product). In other words, ALP activity can be detected in real time by monitoring changes in fluorescence. In the absence of ALP activity, there will be no change in fluorescence. If a compound inhibits ALP activity, then there will be no change in fluorescence.

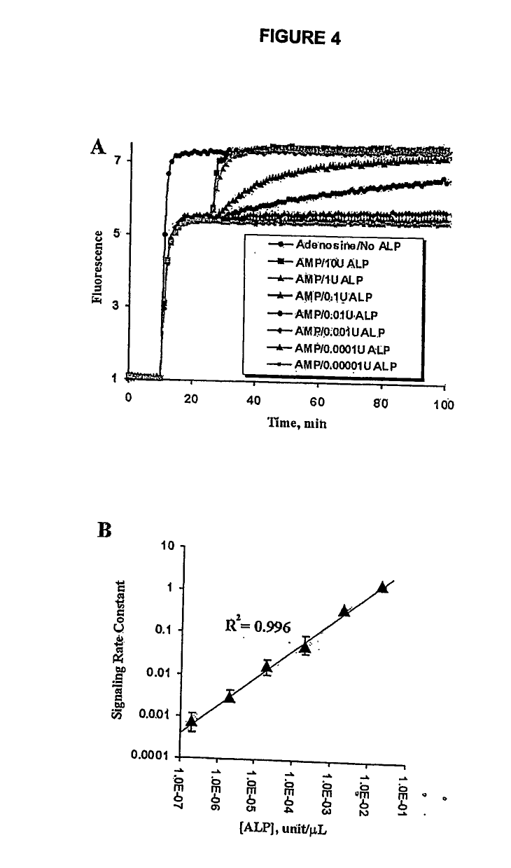

The effect of enzyme concentration on the second leg signaling responses was determined in the presence of 0.75 mM AMP and variable amounts of calf intestine ALP. In total, seven different amounts of ALP were tested. The ALP samples were added to a 500 μL aptamer-AMP complex at the 25th minute as shown in FIG. 4A. The ALP amounts ranged from 10 to 10⁻⁵ units with progressive 10-fold dilution steps. From these tests, the signaling rate constant (reflecting the speed of fluorescence intensity increase in the initial linear signaling range following the ALP addition as a way to measure the enzyme activity) was determined as a function of effective ALP concentration. The results are shown in FIG. 4B. A linear relationship was observed when the enzyme concentration was varied over 6 orders of magnitude. These data clearly demonstrate indicate that the reporter can be utilized as a sensitive probe to quantify effective ALP concentrations and that the methods of the present invention are broadly useful to quantitate enzyme activities. While ALP is used as an example it is clearly apparent that other enzymes can be measured in the same manner.

As discussed above, the present invention also provides a method and means for screening for inhibitors of a chemical reaction. As a model system, the ATP structure switching aptamer complex and the AMP—adenosine reaction were used to demonstrate the utility of the method of the invention and the utility of structure-switching signaling aptamers as unique reporting probes for screening small molecules as enzyme inhibitors.

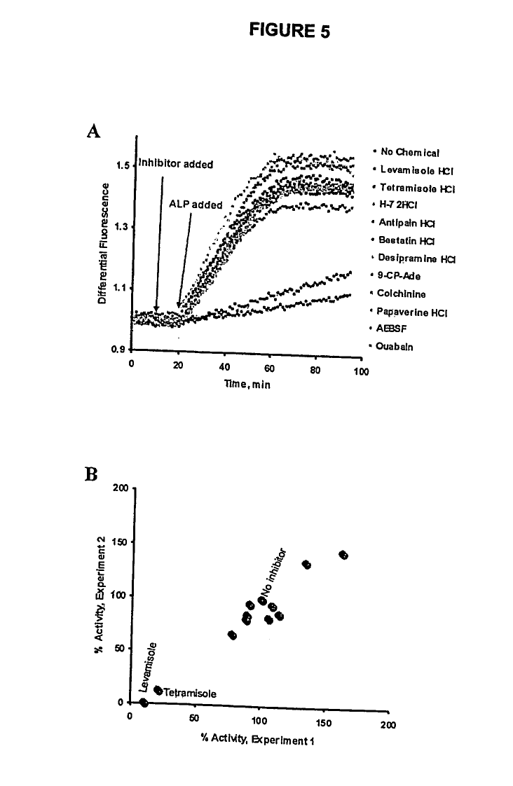

Levamisole and its racemic mixture tetramisole are known inhibitors of porcine ALP (as well as other mammalian ALPs but not calf intestine ALP) with Kᵢ in the low mM range. This suggested that if an appropriate amount of levamisole or tetramisole was added into the anti-ATP signaling aptamer mixture containing AMP, the activity of porcine ALP should be attenuated and this would lead to a slower rate of fluorescence intensity increase in the signaling aptamer solution. To test this, 0.1 mM levamisole or tetramisole was added to the signaling aptamer mixture containing 0.75 mM AMP. The rate of transformation of AMP to adenosine promoted by porcine ALP was considerably reduced relative to the uninhibited reaction as shown in FIG. 5A. As controls, nine other randomly chosen non-inhibiting chemical compounds were also tested at the same concentration. The chemical structures of the small molecules used for screening are shown below. 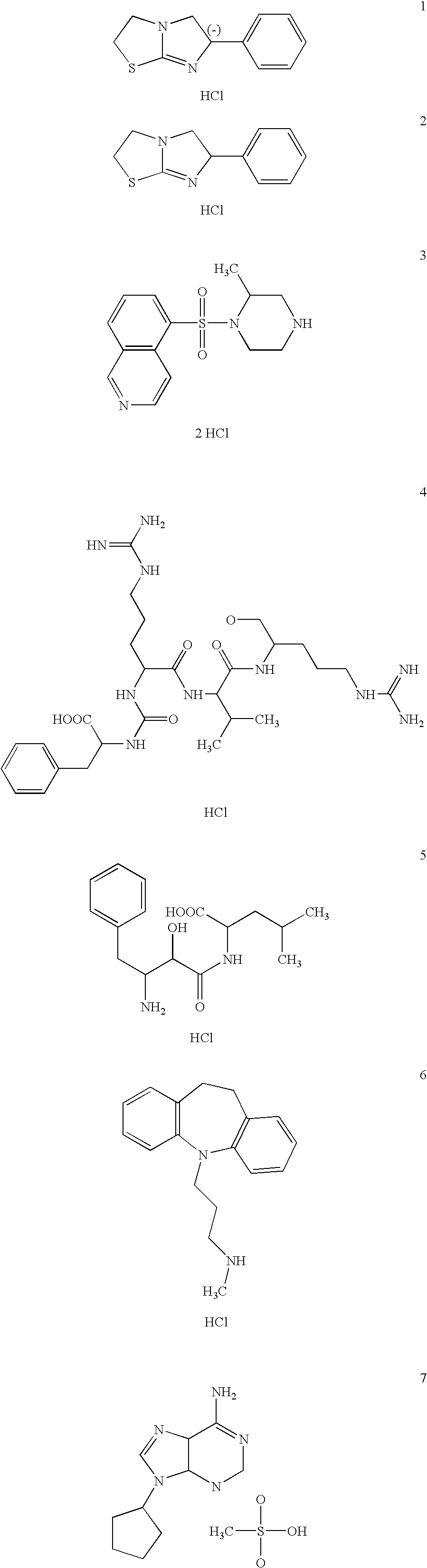 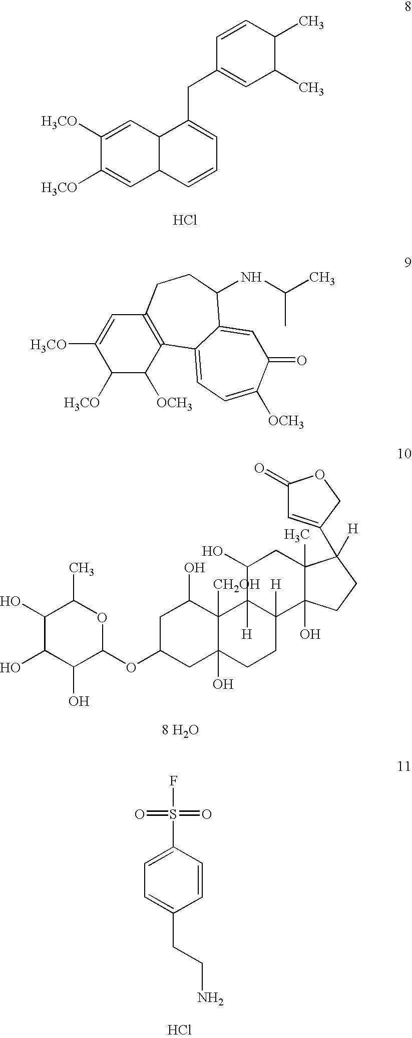
The chemical names of the small molecules are as follows:
1. Levamisole: (−) 2,3,5,6-Tetrahydro-6-phenylimidazo[2,1-b]thiazole hydrochloride;
2. Tetramisole: (±) 2,3,5,6-Tetrahydro-6-phenylimidazo[2,1-b]thiazole hydrochloride;
3. H-7 Dihydrochloride: 1-(5-lsoquinolinylsulfonyl)-2-methylpiperazine dihydrochloride;
4. Antipain Hydrochloride: N-[Nα-carbonyl-Arg-Val-Arg-al]-Phe hydrochoride;
5. Bestatin Hydrochloride: [(2S, 3R)-3-amino-2-hydroxy-4-phenylbutanoyl]-L-leucine hydrochloride;
6. Desipramine Hydrochloride: 10,11-Dihydro-5-(3-(methylamino)propyl)-5H-dibenz(b,f)azepine hydrochloride;
7. 9-CP-Ade: 9-Cyclopentyladenine;
8. Colchicine: (S)-N-(5,6,7,9-Tetrahydro-1,2,3,10-tetramethoxy-9-oxobenzo[a]heptalen-7-yl)acetamide;
9. Papaverine Hydrochloride: 6,7-Dimethoxy-1-(3,4-dimethoxybenzyl)isoquinoline hydrochloride;
10. AEBSF: 4-(2-Aminoethyl)benzenesulfonyl fluoride hydrochloride;
11. Ouabain: Acocantherin. As expected, no significant rate reduction was observed for these compounds.

Using the same eleven compounds, the inhibition assay was performed in a 96-well plate in order to demonstrate the feasibility of exploring structure-switching signaling aptamers for high-throughput screening of small molecules as enzyme inhibitors (FIG. 5B). A mixture was produced for each well that contained the anti-ATP signaling aptamer, AMP (0.75 mM) and one of the eleven compounds (0.1 mM). Two fluorescence readings were taken from each well: the first was recorded when the porcine ALP was added (Fᵢₙᵢₜ) and the second was recorded 60 minutes later (F_{final}). A control well containing the signaling aptamer and AMP but lacking any test compound was also examined. The inhibition effect exhibited by each compound (or the residue activity of the enzyme, % Activity; FIG. 5B) was then calculated by the following equation: (F_{final}/Fᵢₙᵢₜ)_{compound}/(F_{final}/Fᵢₙᵢₜ)_{control}. The results shown in FIG. 5B clearly indicate that structure-switching signaling aptamers and methods of the present invention are suitable for high throughput screening.

The results shown in the model system demonstrate that, based on the ability to distinguish between adenosine, AMP, ADP and ATP, a signaling aptamer complex can be used as a unique reporter to monitor the catalytic activities of nucleotide-dephosphorylating enzymes such as alkaline phosphatase.

While adenosine has been used as an exemplary product in these experiments, it is clearly apparent that the conversion of other substrate targets to product targets as well as intermediate targets can be detected using the method of the present invention. It is also apparent that reporter molecules other than those generating a fluorescent signal can be used i.e. calorimetric, radioactive, etc. . . . ).

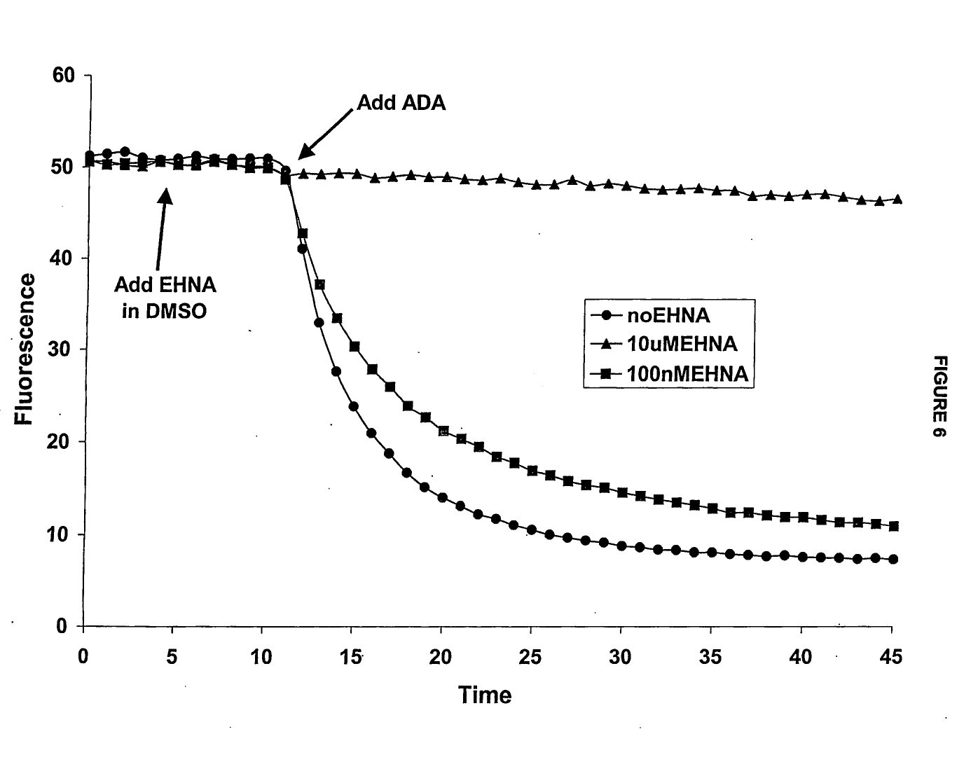

To further demonstrate the broad applicability of the method, a different enzyme system was assessed. Adenosine deaminase transforms adenosine into inosine. The signaling ATP structure switching aptamer complex has virtually no affinity for inosine. Thus conversion from adenosine to inosine results in a decrease in fluorescent signal. This model system was used to demonstrate that inhibitors of adenosine deaminase can be detected using the method of the present invention. EHNA is a known inhibitor of adenosine deaminase. As shown in FIG. 6, the rate of deamination is reduced in the presence of 100 mM EHNA and is virtually stopped in the presence of 10 μM EHNA.

Depending on the enzyme and substrates involved, conversion of a substrate to a product may be detected as an increase or a decrease in fluorescence. It is also possible to follow the enzymatic activity as a substrate or substrate target is converted to an intermediate target and then a product target.

The present invention can also be used to measure the opposite reaction. For example, rather than measuring phosphatase activity, a phosphorylation reaction can be monitored.

It is clearly apparent that the methods of the present invention can be used to monitor a variety of chemical reactions in real time. The methods are especially useful to monitor stages in a signaling pathways.

The methods of the present invention are generally applicable to a broad range of biological and chemical systems for kinetic and mechanistic studies. The components of the assay system can be provided as a kit for the detection of enzyme activity. A kit for detecting modification of a substrate includes a signaling aptamer and a substrate. The kit may optionally include an enzyme control. A kit for screening for inhibitors includes a substrate, a signaling aptamer and an enzyme.

A large number of structure-switching aptamer reporters can be created by the combination of in vitro selection and aptamer engineering for use in the method of the present invention to monitor a chemical reaction of interest. In addition, several structure-switching signaling aptamers carrying different fluorophores or quenchers can be conveniently created. These aptamer reporters can be used to set up various forms of multiplexed assays for real-time monitoring of either multi-step enzymatic reactions or different enzymatic activities in the same solution.

The two-leg fluorescence-based assay can be equally effective even for the compounds with fluorescence-quenching (Papaverine or Desipramine) or enhancing (9-CP-Ade) properties.

The present invention demonstrates that structure-switching signaling aptamers can be used as reporters for chemical reactions in real time. Novel methods that exploit signaling aptamers as fluorescent reagents for reporting a chemical reaction in real time are provided. In addition, the present invention provides methods to quantify enzyme activities with a very large detection dynamic range and very low detection limit. The invention provides for the use of structure-switching signaling aptamers as enzyme-mediated chemical reaction reporters that are compatible with high-throughput screening technology for the identification of enzymatic inhibitors. Moreover, the reporting system of the present invention has a built-in checking mechanism (two-leg signal profiling) for quality control that reduces the chance of reporting false positives.

It is clearly apparent that, while an existing DNA aptamer was used to report a series of different reactions for exemplary purposes, the method could be applied to any other enzymatic transformations for which a differential signaling aptamer can be obtained. The broad applicability of the methods is readily inferred due to the fact that in vitro selection allows convenient selection of DNA aptamers with the ability to distinguish structurally similar compounds, and the structure-switching approach allows easy design of signaling aptamer reporters with large signaling magnitudes. For example, the above anti-ATP signaling aptamer can be used to perform screening assays to search inhibitors for several important enzymes such as adenosine deaminases (which transform adenosine into inosine for which the aptamer has no affinity at all), adenyl cyclases (that transform ATP into cAMP for which the ATP aptamer has very low affinity) or phosphodiesterases (that transform cAMP into AMP). In addition, several structure-switching signaling aptamers carrying different fluorophores or quenchers can be generated through the combination of in vitro selection and signaling aptamer engineering. These aptamers could be used to establish various forms of multiplexed assays for real-time monitoring of either multi-step enzymatic reactions or different enzymatic activities occurring in the same solution. It is clearly apparent that other signaling aptamers that have differential affinities for substrates and products can be used to monitor chemical reactions in real time and to screen for inhibitors of the reaction.

The above disclosure generally describes the present invention. It is believed that one of ordinary skill in the art can, using the preceding description, make and use the compositions and practice the methods of the present invention. A more complete understanding can be obtained by reference to the following specific examples. These Examples are described solely to illustrate preferred embodiments of the present invention and are not intended to limit the scope of the invention. Changes in form and substitution of equivalents are contemplated as circumstances may suggest or render expedient. Other generic configurations will be apparent to one skilled in the art. All journal articles and other documents, such as patents or patent applications, referred to herein are hereby incorporated by reference.

### EXAMPLES

Although specific terms have been used in these examples, such terms are intended in a descriptive sense and not for purposes of limitation. Methods of molecular biology and chemistry referred to but not explicitly described in the disclosure and these examples are reported in the scientific literature and are well known to those skilled in the art.

### Example 1

### DNA Oligonucleotides and Chemical Reagents

Both standard and modified DNA oligonucleotides were prepared by automated DNA synthesis using cyanoethyl-phosphoramidite chemistry (Keck Biotechnology Resource Laboratory, Yale University; Central Facility, McMaster University). 5′-Fluorescein and 3′-DABCYL (4-(4-dimethylamino-phenylazo)benzoic acid) moieties (in FDNA and QDNA, respectively) were introduced using 5′-fluorescein phosphoramidite and 3′-DABCYL-derivatized controlled pore glass (CPG) (Glen Research, Sterling, Va.) and were purified by reverse phase HPLC. HPLC separation was performed on a Beckman-Coulter HPLC System Gold with a 168 Diode Array detector. The HPLC column was an Agilent Zorbax ODS C18 Column, with dimensions of 4.5 mm×250 mm and a 5-□m bead diameter. A two-solvent system was used for the purification of all DNA species, with solvent A being 0.1 M triethylammonium acetate (TEM, pH 6.5) and solvent B being 100% acetonitrile. The best separation results were achieved by a non-linear elution gradient (10% B for 10 min, 10% B to 40% B over 65 min) at a flow rate of 0.5 mL/min. It was found that the main peak had very strong absorption at both 260 nm and 491 nm. The DNA within ⅔ of the peak-width was collected and dried under vacuum. Unmodified DNA oligonucleotides were purified by 10% preparative denaturing (8 M urea) polyacrylamide gel electrophoresis (PAGE), followed by elution and ethanol precipitation. Purified oligonucleotides were dissolved in water and their concentrations were determined spectroscopically.

Calf intestine alkaline phosphatase (calf intestine ALP) was purchased from MBI-Fermentas and porcine ALP from kidney was purchased from Sigma. Both were used without further purification. Adenosine 5′-triphosphate (ATP), adenosine 5′-diphosphate (ADP), adenosine 5′-monophosphate (AMP), and adenosine were purchased from Sigma and their solution concentrations were determined by standard spectroscopic methods. All other chemical reagents were also obtained from Sigma.

### Example 2

### General Procedures for Fluorescence Measurements

The following concentrations of oligonucleotides were used for fluorescence measurements (DNA sequences are shown in FIG. 1A): 40 nM for FDNA, 80 nM for the aptamer (MAP) and 120 nM for the quencher (QDNA). The ratio of FDNA:MAP:QDNA was set to be 1:2:3 to ensure a low background signal. Under this setting, the vast majority of FDNA molecules would form a duplex structure with MAP and the resulting FDNA-MAP duplexes would also be able to engage a QDNA molecule for fluorescence quenching. The assay buffer contained 300 mM NaCl, 5 mM MgCl₂ and 20 mM Tris·HCl (pH 8.3). The fluorescence intensities were recorded on a Cary Eclipse Fluorescence Spectrophotometer (Varian) with excitation at 490 nm and emission at 520 nm. The sample volume in all cases was 500 μL except that 150 μL was used in the 96-well microplate based assay. Measurements of fluorescence intensities from specific samples are detailed below.

### Example 3

### Generation of a Two Leg Signal Upon Target Addition Followed by Enzyme Addition

495 μL of FDNA-QDNA-MAP signaling mixture in the assay buffer was incubated in the absence of any target for 10 min at 22° C., followed by the addition of adenosine (filled green triangles), AMP (filled red circles), ADP (filled purple triangles), and ATP (filled blue squares) to a final concentration of 0.75 mM (achieved using 100× stock); water was added into the control sample (open circles). The resultant aptamer-target mixtures were incubated at the same temperature for 10 more minutes. At this point, 0.5 units of calf intestine ALP were introduced (affording a final concentration of 0.001 unit/μL), and the resultant solution was further incubated for 50 additional minutes. A fluorescence reading was recorded every minute. The raw fluorescence data are shown in FIG. 1B.

### Example 4

### Comparison of Fluorescent Signals for Adenosine AMP and AMP+ALP

Fluorescence readings were taken at 22° C. for FDNA-QDNA-MAP solutions that contained either adenosine (red circles) or AMP (green sequences) at 0, 0.1, 0.25, 0.5, 0.75, 1, or 3 mM. Each experiment was done in triplicate to obtain an average reading. The results are shown in FIG. 1C. A fluorescence differential, ΔF, defined as F_{adenosine}-F_{AMP}, was calculated for each concentration. ΔF is plotted as a function of the target concentration as shown in FIG. 2. Each AMP solution was also treated with 5 units of calf intestine ALP (affording a final concentration of 0.01 unit/μL) for 15 minutes and the resultant solutions were measured again for fluorescence intensity (blue triangles in FIG. 1C). The new measurement afforded readings that matched those of the solutions that contained adenosine at the same concentrations (red circles in FIG. 1C). This indicates that all AMP molecules were converted into adenosine. Each adenosine solution was also treated with calf intestine ALP, which did not induce any fluorescence intensity change as expected (data not shown).

### Example 5

### Monitoring ALP Activity in the Transition from AMP to Adenosine

Dephosphorylation of adenosine monophosphate (AMP) by alkaline phosphatase (AP) was used as a model reaction. A structure-switching signaling DNA aptamer,¹ which exhibits a moderate affinity for the substrate (AMP) and a higher affinity for the product (adenosine), was used as the reporter. A unique two-phase signaling profile can be obtained with such a system. A first signal is produced upon the addition of the substrate target, indicative of the formation of substrate-aptamer complex and a second signal is generated upon the addition of the enzyme, reporting the conversion of AMP to adenosine. The reaction is shown in FIG. 3.

The same procedures described for Example 4 were used to obtain the data shown in FIG. 4 with three variations: (1) only 0.75 mM AMP was used as the target, (2) calf intestine ALP was used at seven different amounts from 10⁻⁵-10¹ units (affording final concentrations of 2×10⁻⁸−2×10⁻² unit/μL), and (3) the incubation time after calf intestine ALP addition was extended to 1200 minutes. Each experiment was done in quadruplicate; however only one set of data is shown.

### Example 6

### Screening of Small Molecules as Enzyme Inhibitors

The ATP reporter and 0.75 mM AMP (500 μL) were incubated for 10 min at room temperature before a test compound was added at a final concentration of 0.1 mM. The resulting mixture was incubated for another 10 min, followed by the addition of 5 units of porcine ALP (resulting in a concentration of 0.01 units/μL) and further incubation for 75 more minutes. The fluorescence intensity (F_{ALP}) was monitored continuously. For the same compound, the fluorescence intensity (F_{NoALP}) of a control reaction with the addition of porcine ALP was also recorded in the same way. FIG. 5A plots F_{ALP}/F_{NoALP} vs. reaction time for eleven compounds tested.

### Example 7

### Inhibition Assay in Micro-Well Plate

The experiment procedure described in Example 6 was performed, in duplicate, in a 96-well plate (150 μL solution was used for each well). The concentrations of all components remained the same. The initial fluorescence intensity (Fᵢₙᵢₜ) was taken when porcine ALP was added. The fluorescence intensity (F_{final}) was again recorded 60 minutes after the addition of the ALP. For each tested compound, an F_{final}/Fᵢₙᵢₜ value was calculated. The inhibition effect (% ALP activity) was calculated as (F_{final}/Fᵢₙᵢₜ)_{compound}/(F_{final}/Fᵢₙᵢₜ)control. The control reaction contained no tested compound. The results are shown in FIG. 5B.

### Example 8

### Adenosine Deaminase Activity

To demonstrate the versatility of the methods to monitor enzymatic reactions generally, adenosine deaminase activity was monitored. Three solutions of 500 μL containing 20 nM fluorescent aptamer, 40 nM antisense DNA labelled with the quencher (called QDNA), 1 mM Adenosine, 300 mM NaC1, 5 mM MgCl₂ and 25 mM HEPES pH 8.0 were equilibrated at 22° C. for the first 5 minutes. Fluorescence reading, made each minute, reveal steady high fluorescence levels corresponding to the complex state of the aptamer with adenosine. After 3 minutes, different concentrations of EHNA.HC1, an adenosine deaminase inhibitor, in DMSO were introduced: no inhibitor (blue line), 100 nM inhibitor (green line) and 10 μM inhibitor (red line). The solutions were further incubated for 5 more minutes. No changes in fluorescence can be observed, suggesting that the inhibitor or DMSO has no influence on the state of the aptamer. Finally, the solutions were exposed to an enzyme, called Adenosine Deaminase (ADA), which transforms adenosine into inosine. The aptamer has no affinity for the inosine, so the transformation should induce the disruption of the aptamer—adenosine complex state and the formation of a duplex state with the QDNA. The phenomenon can be monitored by following the decrease in fluorescence levels due to fluorescence quenching (blue line). However, when the inhibitor of ADA is present, the observed rate of deamination is slower at 100 nM EHNA.HC1 and almost no deamination is observed at 10 μM EHNA.HC1.

### REFERENCES

(1) Nutiu, R.; Li, Y. J. Am. Chem. Soc. 2003, 125, 4771.
(2) Schultz, P. G.; Lerner, R. A. Science 1995, 269, 1835.
(3) Lorsch, J. R.; Szostak, J. W. Acc. Chem. Res. 1996, 29, 103.
(4) Li, Y.; Breaker, R. R. Curr. Opin. Struct. Biol. 1999, 9, 315.
(5) Tuerk, C.; Gold, L. Science, 1990, 249, 505.
(6) Ellington, A. D.; Szostak, J. W. Nature, 1990. 346, 818.
(7) Famulok, M.; Mayer, G.; Blind, M. Acc Chem Res, 2000, 33, 591.
(8) Wilson, D. S.; Szostak, J. W. Annu. Rev. Biochem. 1999. 68, 611.
(9) Jhaveri, S. et al. J. Am. Chem. Soc. 2000. 122, 2469.
(10) Jhaveri, S.; Rajendran, M.; Ellington, A. D. Nat. Biotechnol. 2000, 18, 1293.
(11) Hamaguchi, N.; Ellington, A.; Stanton, M. Anal. Biochem. 2001,294, 126.
(12) Yamamoto, R.; Baba, T.; Kumar, P. K. Genes Cells, 2000, 5, 389.
(13) Li, J. J.; Fang, X.; Tan, W. Biochem. Biophys. Res. Commun. 2002, 292, 31.
(14) Stojanovic, M. N.; de Prada, P.; Landry, D. W. J. Am. Chem. Soc. 2001, 123, 4928.
(15) Stojanovic, M. N.; de Prada, P.; Landry, D. W. J. Am. Chem. Soc. 2000, 122, 11547.
(16) Huizenga, D. E.; Szostak, J. W. Biochemistry, 1995, 34, 656.

1. A method of monitoring a chemical reaction in which substance A is converted to product B, said method comprising: incubating substance A in the presence of a signaling aptamer that has a first affinity for substance A and a second, different affinity for product B, determining the amplitude of the signal based on the affinity of the aptamer for substance A and monitoring for a change in amplitude of the signal wherein a change in amplitude of the signal is indicative of a modification of substance A whereby binding of the signaling aptamer to substance A is disrupted.

2. (canceled)

3. A method according to claim 1, wherein an increase in the amplitude of the signal is indicative of binding of the aptamer to product B.

4. A method according to claim 1, wherein a decrease in the amplitude of the signal is indicative of binding of the aptamer to product B.

5. A method according to claim 1, wherein the signaling aptamer has afluorophore and a quencher in proximity.

6. A method according to claim 5, wherein the signaling aptamer is a signaling aptamer complex (SAC) comprising an aptamer oligonucleotide and a quencher modified oligonucleotide capable of forming a duplex with the aptamer oligonucleotide in the absence of an aptamer binding target.

7. A method according to claim 1, wherein the chemical reaction is addition of a functional group to substance A.

8. A method according to claim 1, wherein the chemical reaction is removal of a functional group from substance A.

9. A method according to claim 1, wherein the chemical reaction is a phosphorylation reaction.

10. A method according to claim 1, wherein substance A is a substrate for an enzyme and product B is a product of an enzymatic reaction.

11. A method according to claim 10, wherein the substrate is selected from the group consisting of inosine, adenosine, cAMP, AMP, ADP and ATP.

12. A method according to claim 10, wherein the enzyme is selected from the group consisting of a phosphatase, a deaminase, an adenyl cyclase and a phosphodiesterase.

13. A method of detecting the presence of an enzyme capable of converting a substrate to a product in a test sample, said method comprising: incubating the substrate with a signaling aptamer that has a different affinities for the substrate and the product in the presence of the test sample and monitoring for a change in signal, wherein a change in signal intensity indicates enzymatic activity in the test sample.

14. A method according to claim 13, wherein an increase in signal intensity indicates the presence of the enzyme.

15. A method according to claim 13, wherein a decrease in signal intensity indicates the presence of the enzyme.

16. A method of quantitating an enzyme in a sample, said method comprising incubating a substrate with a signaling aptamer in the presence of the sample, measuring the amplitude of the signal generated and comparing the amplitude of the signal to a standard curve of signal relative to enzyme concentration.

17. A method of screening a test compound for inhibition of an enzyme, said method comprising: incubating a substrate with a signaling aptamer that has a first affinity for the substrate and a second, different affinity for product, in the presence of the test compound and the enzyme; and monitoring for a change in amplitude of the signal, wherein a change in signal is indicative of enzyme activity and no change is indicative of inhibition of the enzyme.

18. A method according to claim 17, wherein the enzyme is selected from the group consisting of a phosphatase, a deaminase, an adenyl cyclase and a phosphodiesterase.

19. An enzyme inhibitor identified according to the method of claim 17.

20. A kit for detecting modification of a substrate, said kit comprising a substrate and a signaling aptamer having an affinity for the substrate, wherein the signaling aptamer has a different affinity for modified substrate.

21. A kit for screening for enzyme inhibitors, said kit comprising a substrate, an enzyme capable of acting on the substrate to produce a product, and a signaling aptamer having a first affinity for the substrate and a second affinity for the product.
